(19) United States
(12) Patent Application Publication
(10) Pub. No.: US 2004/0259861 A1
(43) Pub. Date: Dec. 23, 2004

(54) Medicament inhibiting sodium/calcium exchange system

(76) Inventor: Naoya Satoh, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/484,463

(22) PCT Filed: Jul. 24, 2002
(86) PCT No.: PCT/JP02/07486

(30) Foreign Application Priority Data
Jul. 25, 2001 (JP) 2001-224916

(51) Int. Cl. A61K 31/55; A61K 31/495; A61K 31/4164

(52) U.S. Cl. 514/218; 514/255.03; 514/386

(57) ABSTRACT

A medicament for inhibiting sodium/calcium exchange system which comprises as an active ingredient a substance selected from the group consisting of an aminobenzenesulfonic acid derivative represented by the following general formula (I) and a salt thereof, and a hydrate thereof and a solvate thereof:

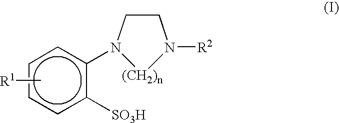

wherein R¹ represents a hydrogen atom, a C₁-C₆ alkyl group and the like; R² represents a hydrogen atom, a C₁-C₆ alkyl group, or a C₇-C₁₂ aralkyl group; and n represents an integer of from 1 to 4. The medicament is useful for suppression of intracellular calcium accumulation generated under very severe dyscrasic condition as a result of clinically-occurred combination of ischemia/reperfusion and intracellular sodium increase.

### TECHNICAL FIELD

The present invention relates to a medicament which inhibits sodium/calcium exchange system.

### BACKGROUND ART

In ischemic heart disease such as myocardial infarction and angina pectoris, coronary blood flow blocking for a certain period of time and resumption of coronary blood flow by the recanalization therapy will occur. It is known that, following the recanalization, a rapid calcium ion inflow arises intracellularly from outside of myocardium cells, and subsequently, through various calcium dependent reactions such as activation of a calcium dependent protease, activation of a calcium dependent lipid decomposition enzyme, and reduction of energy generation, irreversible cardiomyopathy is caused. It is considered that the calcium inflow is based on sodium flow into the cells, which conjugates with extracellular excretion of protons accumulated in the cells during the ischemia and occurs through sodium/proton exchange system, and is also based on calcium flow into the cells, which conjugates with extracellular excretion of sodium in the cells and occurs through sodium/calcium exchange system.

As far as the inventors of the present invention are aware, no agent is known at present which suppresses intracellular calcium accumulation that is generated under very severe dyscrasic conditions as a result of combination of the clinically-occurred ischemia/reperfusion and the increase of intracellular sodium.

Aminobenzenesulfonic acid derivatives which have suppressing action on hyper intracellular accumulation of calcium ion in myocardium cells or vessel smooth muscles are known (Japanese Patent Unexamined Publication (KOKAI) No. 3-7263). As for these compounds, it is known that the compounds are potentially useful as preventive and therapeutic medicaments for ischemic heart disease, heart failure, hypertension, arrhythmia and the like based on suppression or reduction of cardiomyopathy, dysfunction of heart conduction and the like, without β receptor stimulant-like action, β receptor blocker-like action, or calcium channel antagonist-like action (Japanese Patent Unexamined Publication (KOKAI) No. 3-7263 and Japanese Patent Unexamined Publication (KOKAI) No. 4-139127). Japanese Patent Unexamined Publication (KOKAI) No. 10-298077 discloses that the aforementioned compounds have remarkable improving effect on heart hypofunction under pathological cardiomyopathy, and also have improving effect on long-term survival rate of idiopathic cardiomyopathy to achieve prolongation of life of a patient. In addition, International Publication WO 99/40919 discloses that the aforementioned compounds have promoting effect on calcium ion uptake by myocardium sarcoplasmic reticulum, and are useful for therapeutic treatment or prevention of dysfunction of dilatation of heart.

However, these publications fail to disclose whether the aforementioned compounds suppress intracellular calcium accumulation that is generated under very severe dyscrasic conditions as a result of clinically-occurred combination of the ischemia/reperfusion and the intracellular sodium increase. It is already known that the aforementioned compounds suppress the increase of myocardium calcium content resulting from ischemia/reperfusion (calcium overload). However, it has not yet been known to date whether or not the aforementioned compounds suppress calcium increase even under severe dyscrasic conditions such as mentioned above.

### DISCLOSURE OF THE INVENTION

An object of the present invention is provide a medicament which suppresses intracellular calcium accumulation that is generated under a severe dyscrasic condition as a result of clinically-occurred combination of ischemia/reperfusion and intracellular sodium increase.

The inventors of the present invention conducted various studies to achieve the foregoing object. As a result, they found that specific aminobenzenesulfonic acid derivatives or salts thereof, or hydrates thereof or solvates thereof had inhibitory action against sodium/calcium exchange system, and based on said action, the substances suppressed intracellular calcium accumulation generated under severe dyscrasic conditions as a result of clinically-occurred combination of ischemia/reperfusion and intracellular sodium increase.

The present invention thus provides a medicament for inhibiting sodium/calcium exchange system which comprises as an active ingredient a substance selected from the group consisting of an aminobenzenesulfonic acid derivative represented by the following general formula (I) and a salt thereof, and a hydrate thereof and a solvate thereof:

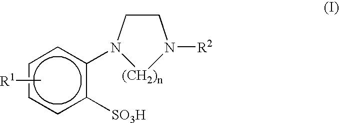

wherein R¹ represents a hydrogen atom, a C₁-C₆ alkyl group, a C₃-C₇ cycloalkyl group, a halogenated C₁-C₄ alkyl group, a halogen atom, or a C₆-C₁₂ aryl group; R² represents a hydrogen atom, a C₁-C₆ alkyl group, or a C₇-C₁₂ aralkyl group which may have one or more substituents selected from the group consisting of cyano group, nitro group, a C₁-C₆ alkoxyl group, a halogen atom, a C₁-C₆ alkyl group, and amino group; and n represents an integer of from 1 to 4.

As a preferred embodiment of the present invention, provided are the aforementioned medicament for therapeutic and/or preventive treatment of dysfunction resulting from ischemia/reperfusion; the aforementioned medicament for suppressing increase in myocardium calcium content induced by dysfunction resulting from ischemia/reperfusion; and the aforementioned medicament for suppressing increase of myocardium calcium content which is generated under condition of a combination of ischemia/reperfusion and increase of myocardium sodium content.

From another aspect, provided is an inhibitor against sodium/calcium exchange system which comprises a substance selected from the group consisting of an aminobenzenesulfonic acid derivative represented by the above general formula (I) and a salt thereof, and a hydrate thereof and a solvate thereof.

From further aspect, provided is a use of a substance selected from the group consisting of an aminobenzenesulfonic acid derivative represented by the above general formula (I) and a salt thereof, and a hydrate thereof and a solvate thereof for manufacture of the aforementioned medicament.

From still further aspect, provided are:

a method for inhibition of sodium/calcium exchange system which comprises the step of administering to a mammal including human an effective amount of a substance selected from the group consisting of an aminobenzenesulfonic acid derivative represented by the above general formula (I) and a salt thereof, and a hydrate thereof and a solvate thereof:

a method for therapeutic and/or preventive treatment of a dysfunction resulting from ischemia/reperfusion, which comprises the step of administering to a mammal including human a therapeutically and/or preventively effective amount of a substance selected from the group consisting of an aminobenzenesulfonic acid derivative represented by the above general formula (I) and a salt thereof, and a hydrate thereof and a solvate thereof:

a method for suppressing increase of myocardium calcium content induced by a dysfunction resulting from ischemia/reperfusion, which comprises the step of administering to a mammal including human an effective amount of a substance selected from the group consisting of an aminobenzenesulfonic acid derivative represented by the above general formula (I) and a salt thereof, and a hydrate thereof and a solvate thereof: and

a method for suppressing increase of myocardium calcium content which is generated under condition of a combination of ischemia/reperfusion and increase of myocardium sodium content, which comprises the step of administering to a mammal including human an effective amount of a substance selected from the group consisting of an aminobenzenesulfonic acid derivative represented by the above general formula (I) and a salt thereof, and a hydrate thereof and a solvate thereof.

### BEST MODE FOR CARRYING OUT THE INVENTION

The medicament of the present invention comprises a substance selected from the group consisting of an aminobenzenesulfonic acid derivative represented by the above general formula (I) and a salt thereof, and a hydrate thereof and a solvate thereof as an active ingredient, and has inhibitory action against sodium/calcium exchange system. As demonstrated by the example given below, the medicament of the present invention is effective for suppressing increase of intracellular calcium content which is generated under very severe dyscrasic conditions as a result of combination of clinically-occurred ischemia/reperfusion and increase of intracellular sodium.

A degree of myocardiopathy resulting from ischemia/reperfusion correlates a period of ischemia. Larger amounts of protons become accumulated in myocardial cells as a metabolic product when ischemia period is prolonged, and a large amount of sodium flows into the myocardial cells as exchange for the protons through sodium/proton exchange system. Subsequently, in exchange for the sodium which is increased intracellularly, a still larger amount of calcium flows into the myocardial cells through sodium/calcium exchange system. Based on the suppressing action against the sodium/calcium exchange system, the medicament of the present invention can suppress the intracellular calcium accumulation generated under the very severe dyscrasic conditions as a result of the combination of the ischemia/reperfusion and the increase of intracellular sodium. Accordingly, even when an ischemia period is prolonged by some reasons such as a delay in patient conveyance to a hospital after an ischemic heart stroke and an unsuccessful treatment of recanalization, the medicament of the present invention can effectively suppress myocardiopathy.

Active ingredients of the medicament of the present invention includes substances selected from the group consisting of aminobenzenesulfonic acid derivatives represented by the following general formula (I) and salts thereof, and hydrates thereof and solvates thereof.

In the aforementioned general formula (I), examples of the C₁-C₆ alkyl group defined by R¹ include methyl group, ethyl group, propyl group, isopropyl group, butyl group, isobutyl group, sec-butyl group, tert-butyl group, pentyl group, isopentyl group, neopentyl group, tert-pentyl group, hexyl group, and isohexyl group. Examples of the C₃-C₇ cycloalkyl group include cyclopropyl group, cyclobutyl group, cyclopentyl group, cyclohexyl group, and cycloheptyl group. Examples of the halogenated C₁-C₄ alkyl group include trifluoromethyl group, trifluoroethyl group, and pentafluoroethyl group. Examples of the halogen atom include fluorine atom, chlorine atom, and bromine atom. Examples of the C₆-C₁₂ aryl group include phenyl group and naphthyl group.

Preferred examples of R¹ include a hydrogen atom, a C₁-C₆ alkyl group, a C₅-C₆ cycloalkyl group, trifluoromethyl group, a halogen atom, or a phenyl group, and more preferred examples include C₁-C₃ alkyl group, cyclohexyl group, trifluoromethyl group, a chlorine atom, a bromine atom, or a phenyl group. R¹ is most preferably methyl group or propyl group.

Examples of the C₁-C₆ alkyl group defined by R² include, for example, alkyl groups defined as for the aforementioned R¹. Examples of the C₇-C₁₂ aralkyl group include, for example, benzyl group, phenethyl group, and naphthylmethyl group. The aralkyl group may have one or more substituents selected from the group consisting of cyano group; nitro group; a C₁-C₆ alkoxyl group such as methoxy group, ethoxy group, propoxy group, isopropoxy group, butoxy group, isobutoxy group, tert-butoxy group, pentyloxy group, isopentyloxy group, tert-pentyloxy group, or hexyloxy group; a halogen atom such as those defined as for the aforementioned R₁; an alkyl group such as those defined as for the aforementioned R₁; and an amino group.

Preferred examples of R² include a hydrogen atom, a C₁-C₃ alkyl group, or a C₇-C₁₂ aralkyl group which may have one or more substituents selected from the group consisting of a C₁-C₃ alkyl group, a C₁-C₃ alkoxyl group, and a halogen atom, and more preferred examples include a hydrogen atom or a C₇-C₁₂ aralkyl group which may have one or more substituents selected from the group consisting of a C₁-C₃ alkoxyl group. R² is most preferably a hydrogen atom.

In the aforementioned general formula (I), symbol n is preferably 2.

Specific examples of the compounds represented by the aforementioned general formula (I), which are preferred as active ingredients of the medicament of the present invention, include the compounds listed in the following tables 1 and 2.

TABLE 1

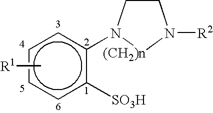

| Compound | Substituting | | | |
|---|---|---|---|---|
| No. | position of R¹ | R¹ | n | R² |
| 1 | — | H | 2 | H |
| 2 | 3 | —CH₃ | 2 | H |
| 3 | 3 | —CH₂CH₃ | 2 | H |
| 4 | 3 | —CH₂CH₂CH₃ | 2 | H |
| 5 | 3 | —CH(CH₃)₂ | 2 | H |
| 6 | 3 | —(CH₂)₃CH₃ | 2 | H |
| 7 | 4 | —CH₃ | 2 | H |
| 8 | 4 | —CH₂CH₃ | 2 | H |
| 9 | 4 | —(CH₂)₂CH₃ | 2 | H |
| 10 | 4 | —CH(CH₃)₂ | 2 | H |
| 11 | 4 | —(CH₂)₃CH₃ | 2 | H |
| 12 | 5 | —CH₃ | 2 | H |
| 13 | 5 | —CH₂CH₃ | 2 | H |
| 14 | 5 | —(CH₂)₂CH₃ | 2 | H |
| 15 | 5 | —CH(CH₃)₂ | 2 | H |
| 16 | 5 | —(CH₂)₃CH₃ | 2 | H |
| 17 | 5 | —(CH₂)₄CH₃ | 2 | H |
| 18 | 5 | —(CH₂)₅CH₃ | 2 | H |
| 19 | 6 | —CH₃ | 2 | H |
| 20 | 6 | —CH₂CH₃ | 2 | H |
| 21 | 6 | —(CH₂)₂CH₃ | 2 | H |
| 22 | — | H | 2 | —CH₃ |
| 23 | 3 | —CH₂CH₃ | 2 | —CH₃ |
| 24 | 3 | —(CH₂)₂CH₃ | 2 | —CH₃ |
| 25 | 3 | —CH(CH₃₎₂ | 2 | —CH₃ |
| 26 | 3 | —(CH₂)₃CH₃ | 2 | —CH₃ |
| 27 | 4 | —CH₃ | 2 | —CH₃ |
| 28 | 4 | —CH₂CH₂ | 2 | —CH₃ |
| 29 | 4 | —(CH₂)₂CH₃ | 2 | —CH₃ |
| 30 | 5 | —CH₃ | 2 | —CH₃ |
| 31 | 5 | —CH₂CH₃ | 2 | —CH₃ |
| 32 | 5 | —(CH₂)₂CH₃ | 2 | —CH₃ |
| 33 | 5 | —CH(CH₃)₂ | 2 | —CH₃ |
| 34 | 5 | —(CH₂)₃CH₃ | 2 | —CH₃ |
| 35 | 5 | —(CH₂)₄CH₃ | 2 | —CH₃ |
| 36 | 5 | —(CH₂)₅CH₃ | 2 | —CH₃ |
| 37 | 6 | —CH₃ | 2 | —CH₃ |
| 38 | 6 | —CH₂CH₃ | 2 | —CH₃ |
| 39 | 6 | —(CH₂)₂CH₃ | 2 | —CH₃ |
| 40 | 6 | —CH(CH₃)₂ | 2 | —CH₃ |
| 41 | 6 | —(CH₂)₃CH₃ | 2 | —CH₃ |
| 42 | 3 | —(CH₂)₂CH₃ | 2 | —(CH₂)₂CH₃ |
| 43 | 4 | —(CH₂)₂CH₃ | 2 | —(CH₂)₂CH₃ |
| 44 | 5 | —CH₃ | 2 | —(CH₂)₂CH₃ |
| 45 | 5 | —CH₂CH₃ | 2 | —(CH₂)₂CH₃ |
| 46 | 5 | —(CH₂)₂CH₃ | 2 | —(CH₂)₂CH₃ |
| 47 | 5 | —CH(CH₃)₂ | 2 | —(CH₂)₂CH₃ |
| 48 | 5 | —(CH₂)₃CH₃ | 2 | —(CH₂)₂CH₃ |
| 49 | 5 | —(CH₂)₅CH₃ | 2 | —(CH₂)₂CH₃ |
| 50 | — | H | 2 | —(CH₂)₂CH₃ |
| 51 | — | H | 2 | 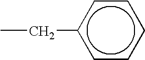 |
| 52 | 3 | —CH₃ | 2 | 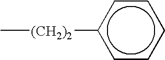 |
| 53 | 3 | —(CH₂)₂CH₃ | 2 | 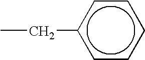 |
| 54 | 4 | —CH₃ | 2 | 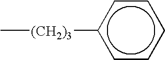 |
| 55 | 4 | —(CH₂)₂CH₃ | 2 | 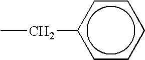 |
| 56 | 5 | —CH₃ | 2 | 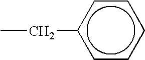 |
| 57 | 5 | —CH₂CH₃ | 2 | 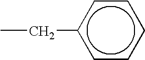 |
| 58 | 5 | —(CH₂)₂CH₃ | 2 | 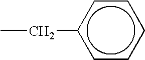 |
| 59 | 5 | —CH(CH₃)₂ | 2 | 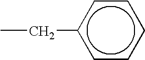 |
| 60 | 5 | —(CH₂)₃CH₃ | 2 | 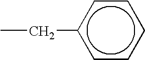 |
| 61 | 5 | —(CH₂)₄CH₃ | 2 | 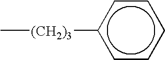 |
| 62 | 5 | —(CH₂)₂CH₃ | 2 | 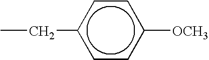 |
| 63 | 5 | —CH(CH₃)₂ | 2 | 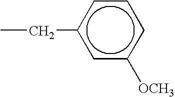 |
| 64 | 5 | —CH(CH₃)₂ | 2 | 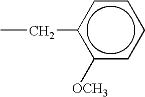 |
| 65 | 4 | —(CH₂)₂CH₃ | 2 | 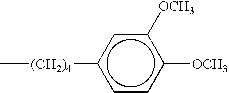 |
| 66 | 5 | —(CH₂)₂CH₃ | 2 | 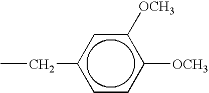 |
| 67 | 5 | —CH(CH₃)₂ | 2 | 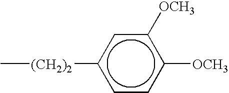 |
| 68 | 6 | —(CH₂)₂CH₃ | 2 | 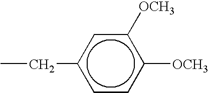 |
| 69 | 5 | —(CH₂)₂CH₃ | 2 | 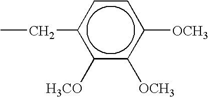 |
| 70 | 6 | —(CH₂)₂CH₃ | 2 | 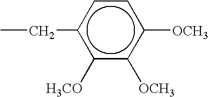 |
| 71 | 3 | —(CH₂)₂CH₃ | 2 | 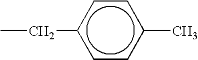 |
| 72 | 4 | —(CH₂)₂CH₃ | 2 | 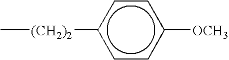 |
| 73 | 5 | —(CH₂)₂CH₃ | 2 | 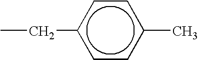 |
| 74 | 6 | —CH(CH₃)₂ | 2 | 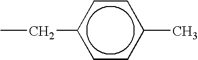 |
| 75 | 3 | —(CH₂)₂CH₃ | 2 | 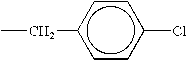 |
| 76 | 4 | —(CH₂)₂CH₃ | 2 | 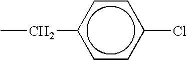 |
| 77 | 5 | —(CH₂)₂CH₃ | 2 | 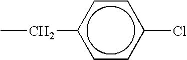 |
| 78 | 6 | —(CH₂)₂CH₃ | 2 | 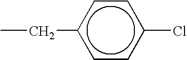 |
| 79 | 3 | —(CH₂)₂CH₃ | 2 | 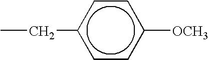 |
| 80 | 4 | —(CH₂)₂CH₃ | 2 | 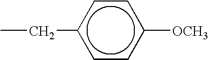 |
| 81 | 5 | —(CH₂)₂CH₃ | 2 | 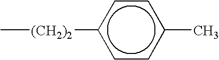 |
| 82 | 6 | —(CH₂)₂CH₃ | 2 | 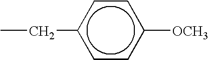 |
| 83 | — | H | 3 | H |
| 84 | 5 | —CH₃ | 3 | H |
| 85 | 5 | —CH₂CH₃ | 3 | H |
| 86 | 5 | —CH₂CH₃ | 3 | H |
| 87 | 5 | —CH(CH₃)₂ | 3 | H |
| 88 | 5 | —(CH₂)₂CH₃ | 3 | H |
| 89 | 5 | —(CH₂)₂CH₃ | 3 | —CH₃ |
| 90 | 5 | —(CH₂)₂CH₃ | 3 | 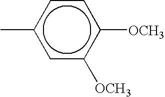 |
| 91 | 5 | 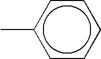 | 2 | H |
| 92 | 5 | —F | 2 | H |
| 93 | 5 | —Cl | 2 | H |
| 94 | 5 | —Br | 2 | H |
| 95 | 5 | —CF₃ | 2 | H |
| 96 | 5 | 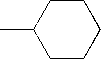 | 2 | H |
| 97 | 5 | 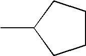 | 2 | H |
| 98 | 5 | 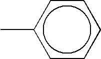 | 2 | —CH₃ |
| 99 | 5 | —Cl | 2 | —CH₃ |
| 100 | 5 | —Br | 2 | —CH₃ |
| 101 | 5 | —CF₃ | 2 | —CH₃ |
| 102 | 5 | 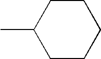 | 2 | —CH₃ |
| 103 | 5 | 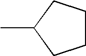 | 2 | —CH₃ |
| 104 | 5 | 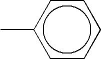 | 2 | 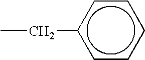 |
| 105 | 5 | —Cl | 2 | 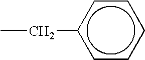 |
| 106 | 5 | —Br | 2 | 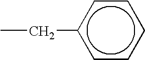 |
| 107 | 5 | —CF₃ | 2 | 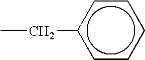 |
| 108 | 5 | 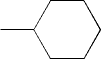 | 2 | 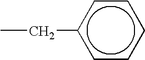 |
| 109 | 5 | 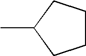 | 2 | 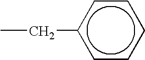 |

TABLE 2

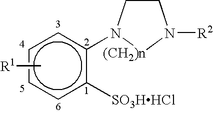

| Compound | Substituting | | | |
|---|---|---|---|---|
| No. | position of R¹ | R¹ | n | R² |
| 110 | 5 | —CH₂CH₂CH₃ | 2 | H |
| 111 | 5 | —CH(CH₃)₂ | 2 | H |
| 112 | 5 | 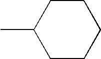 | 2 | H |
| 113 | 5 | 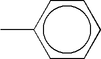 | 2 | H |
| 114 | 5 | —Cl | 2 | H |
| 115 | 5 | —Br | 2 | H |
| 116 | 5 | —CF₃ | 2 | H |

In the tables 1 and 2, the compounds wherein the substituting position is position-5 are preferred, and more preferred compounds include the following compounds:

5-methyl-2-(1-piperazinyl)benzenesulfonic acid;

5-trifluoromethyl-2-(1-piperazinyl)benzenesulfonic acid;

5-n-propyl-2-(1-piperazinyl)benzenesulfonic acid;

5-phenyl-2-(1-piperazinyl)benzenesulfonic acid;

5-chloro-2-(1-piperazinyl)benzenesulfonic acid;

5-bromo-2-(1-piperazinyl)benzenesulfonic acid;

5-isopropyl-2-(1-piperazinyl)benzenesulfonic acid;

5-cyclohexyl-2-(1-piperazinyl)benzenesulfonic acid;

5-n-propyl-2-( 1-homopiperazinyl)benzenesulfonic acid;

5-n-propyl-2-[4-(2 ,3 ,4-trimethoxybenzyl)-1-piperazinyl]benzenesulfonic acid; and

5-n-propyl-2-[4-(3 ,4-dimethoxybenzyl)-1-piperazinyl]benzenesulfonic acid.

Among the aforementioned compounds, most preferable examples include 5-methyl-2-(1-piperazinyl)benzenesulfonic acid and 5-n-propyl-2-(1-piperazinyl)-benzenesulfonic acid.

Pharmaceutically acceptable salts of the aforementioned compounds can also be used as active ingredients of the medicament of the present invention. Examples of salts of the aforementioned compounds include, for example, alkali metal salts and alkaline earth metal salts such as sodium salts, potassium salts, magnesium salts, calcium salts, or aluminum salts; ammonium salts; amine salts such as lower alkylamine salts such as triethylamine salts, hydroxy-lower alkylamine salts such as 2-hydroxyethylamine salts, bis-(2-hydroxyethyl)amine salts, tris(hydroxymethyl)aminomethane salts, or N-methyl-D-glucamine salts, cycloalkylamine salts such as dicyclohexylamine salts, benzylamine salts such as N,N-dibenzylethylenediamine salts or dibenzylamine salts; inorganic acid salts such as hydrochloric acid salts, hydrobromic acid salts, sulfuric acid salts, or phosphoric acid salts; and organic acid salts such as, for example, fumaric acid salts, succinic acid salts, oxalic acid salts, or lactic acid salts.

In addition to the compounds in free form or salts, any hydrates or solvates thereof can also be used as an active ingredient of the medicament of the present invention. Examples of solvents which can form the solvates of the aforementioned compound include, for example, methanol, ethanol, isopropyl alcohol, acetone, ethyl acetate, methylene chloride and the like.

A most preferred example of the active ingredient of the medicament of the present invention includes 5-methyl-2-(1-piperazinyl)benzenesulfonic acid monohydrate.

The aminobenzenesulfonic acid derivatives represented by the aforementioned general formula (I) are known. For example, according to the methods described in Japanese Patent Unexamined Publication (KOKAI) Nos. 3-7263 and 9-221479, European Patent Publication Nos. 390654 and 779283, and U.S. Pat. Nos. 5,053,409 and 5,990,113 and the like, one or ordinary skill in the art can readily synthesize and obtain said compounds.

As the medicament of the present invention, a substance, per se, which is selected from the group consisting of the aminobenzenesulfonic acid derivative represented by the above general formula (I) and a salt thereof, and a hydrate thereof and a solvate thereof can be administered. Alternatively, a pharmaceutical composition comprising the aforementioned substance as an active ingredient and one or more pharmaceutical additive can be prepared and administered.

The medicament of the present invention can be orally or parenterally administered to a mammal including a human. Examples of the forms of pharmaceutical compositions suitable for oral administration include granules, subtilized granules, powders, tablets, hard capsules, soft capsules, syrups, emulsions, suspensions, solutions and the like. Examples of the forms of pharmaceutical compositions suitable for parenteral administration include injections, a suppositories, transdermal preparations and the like.

For manufacture of the aforementioned pharmaceutical compositions, such as a solid or liquid pharmaceutical carriers, or ordinarily used pharmaceutical additives such as excipients, stabilizers, lubricants, sweetening agents, preservatives, suspending aids and the like can be used. A ratio of the active ingredient to the pharmaceutical additive is not particularly limited. For example, the ratio may preferably be 1 to 90% by weight.

Examples of solid pharmaceutical additives include, for example, lactose, kaolin, sucrose, crystalline cellulose, cornstarch, talc, agar, pectin, acacia, stearic acid, magnesium stearate, lecithin, sodium chloride and the like. Examples of liquid carriers include syrup, glycerol, peanut oil, polyvinylpyrrolidone, olive oil, ethanol, benzyl alcohol, propylene glycol, water and the like.

A dose of the medicament of the present invention can be suitably determined depending on, for example, a purpose of treatment or prevention, a kind of a disorder to be treated or prevented, symptoms, body weight, age, and sexuality of a patient, and a kind of the aforementioned substance as an active ingredient. For example, a dose of 0.01 to 1,000 mg per day as the weight of the compound represented by the aforementioned general formula (I) can generally be administered orally to an adult. The above dose may preferably be administered once a day or several times a day as divided portions.

### EXAMPLE

The present invention will be more specifically explained by an example. However, the scope of the present invention is not limited to the example.

In the following example, 5-methyl-2-(1-piperazinyl)benzenesulfonic acid monohydrate was used as the active ingredient of the medicament of the present invention (hereinafter referred to as “the medicament of the present invention”). This substance was prepared according to Example 1 of Japanese Patent Unexamined Publication (KOKAI) No. 9-221479.

### EXPERIMENTAL METHODS

The heart of the rat was excised and perfused with Krebs buffer solution (in mM: NaCl 119, KCl 4.6, MgSO₄.7H₂O 1.2, CaCl₂.2H₂O 1.3, NaHCO₃ 25, KH2PO₄ 1.2, glucose 11; pH 7.4, 37° C.) according to Langendorff method. A thread attached to the apex of the heart was connected to a tension transducer to determine contractile tension. The heart was perfused with a perfusion solution containing monensin (5 μ M; sodium ionophore) for 10 min and then ischemia was induced by a cessation of coronary flow (for 15 min). After reperfusion for 30 min, the heart was liquefied in nitric acid, and ventricular total calcium content was determined by atomic absorption analysis. Contractile tension was measured during the experiment, and recovery in contractile tension at 30 min of reperfusion related to its pre-value was used as an index of cardiac contraction.

### RESULTS

The results are shown in Table 3. In the table, ** represents p<0.01 vs. control by Dunnett's multiple comparison test, and *** represents P<0.001 vs. control by Dunnett's multiple comparison test.

In the heart treated with ischemia/reperfusion in combination with monensin treatment (control), increase in ventricular total calcium content and decrease in recovery in contractile tension were observed compared with those in normoxic hearts (normal). Since the increase in calcium content was dependent on intracellular sodium, the Ca⁺⁺ influx was considered to be mediated by sodium/calcium exchanger. Further, the decrease in recovery in contractile tension was small in the absence of moneisin, suggesting that the decrease was related to the increase in calcium content. The medicament of the present invention improved the increased ventricular calcium content and the decreased recovery in contractile tension which were induced by moneisin treatment and ischemia/reperfusion. Diltiazem (a calcium antagonist; purchased from Sigma) and Amiloride (an inhibitor of sodium/proton exchanger; purchased from Sigma) failed to exhibit these effects.

TABLE 3
The effect of the medicament of the present invention on
ventricular calcium content and recovery in contractile tension

| | | | Recovery in |
|---|---|---|---|
| | | | Contractile |
| | | Calcium Content | Tension |
| Group | N | (μmol/g) | (%) |
| Normal | 8 | 2.15 ± 0.11** | 91.2 ± 2.9*** |
| Without Moneisin | 8 | 2.19 ± 0.13 | 65.0 ± 5.3 |
| Control | 8 | 6.20 ± 0.23 | 5.3 ± 2.1 |
| Compound of the Present | | | |
| Invention 10⁻⁷ M | 8 | 3.72 ± 0.16** | 24.9 ± 4.4** |
| Compound of the Present | | | |
| Invention 10⁻⁶ M | 8 | 2.72 ± 0.18** | 38.5 ± 7.5*** |
| Diltiazem 10⁻⁶ M | 8 | 5.75 ± 0.16 | 11.0 ± 3.3 |
| Diltiazem 10⁻⁵ M | 8 | 5.89 ± 0.22 | 7.4 ± 2.0 |
| Amiloride 10⁻⁴ M | 8 | 5.30 ± 0.25 | 8.9 ± 2.2 |
| Amiloride 10⁻³ M | 8 | 4.26 ± 0.40 | 15.6 ± 2.9 |

From the above results, the medicament of the present invention was demonstrated to be effective in reducing increase in ventricular calcium content induced by sodium overload and ischemia/reperfusion based on inhibition of the sodium/calcium exchanger.

### INDUSTRIAL APPLICABILITY

A novel class of medicament inhibiting sodium/calcium exchange system is provided by the present invention. The medicament of the present invention is effective for suppression of intracellular calcium accumulation generated under very severe dyscrasic condition as a result of clinically-occurred combination of ischemia/reperfusion and intracellular sodium increase.

1. A medicament for inhibiting sodium/calcium exchange system which comprises as an active ingredient a substance selected from the group consisting of an aminobenzenesulfonic acid derivative represented by the following general formula I and a salt thereof, and a hydrate thereof and a solvate thereof: 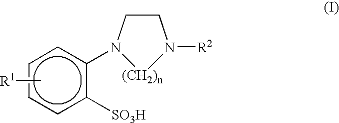
wherein R¹ represents a hydrogen atom, a C₁-C₆ alkyl group, a C₃-C₇ cycloalkyl group, a halogenated C₁-C₄ alkyl group, a halogen atom, or a C₆-C₁₂ aryl group; R² represents a hydrogen atom, a C₁-C₆ alkyl group, or a C₇-C₁₂ aralkyl group which may have one or more substituents selected from the group consisting of cyano group, nitro group, a C₁-C₆ alkoxyl group, a halogen atom, a C₁-C₆ alkyl group, and amino group; and n represents an integer of from 1 to 4.

2. The medicament according to claim 1, which is used for therapeutic and/or preventive treatment of a dysfunction resulting from ischemia/reperfusion.

3. The medicament according to claim 1, which is used for suppressing increase in myocardium calcium content induced by a dysfunction resulting from ischemia/reperfusion.

4. The medicament according to claim 1, which is used for suppressing increase of myocardium calcium content generated under condition of a combination of ischemia/reperfusion and increase of myocardium sodium content.

5. The medicament according to claim 1, wherein substituting position of R¹ is position-5.

6. The medicament according to claim 1, wherein n is 2.

7. The medicament according to claim 1, wherein R² is a hydrogen atom, a C₁-C₃ alkyl group, or a C₇-C₁₂ aralkyl group which may have one or more substituents selected from the group consisting of a C₁-C₃ alkyl group, a C₁-C₃ alkoxyl group, and a halogen atom.

8. The medicament according to claim 1, wherein R² is a hydrogen atom or a C₇-C₁₂ aralkyl group which may have one or more substituents selected from the group consisting of a C₁-C₃ alkoxyl group.

9. The medicament according to claim 1, wherein R² is a hydrogen atom.

10. The medicament according to claim 1, wherein R¹ is a hydrogen atom, a C₁-C₆ alkyl group, a C₅-C₆ cycloalkyl group, trifluoromethyl group, a halogen atom, or a phenyl group.

11. The medicament according to claim 1, wherein R¹is a C₁-C₃ alkyl group, cyclohexyl group, trifluoromethyl group, a chlorine atom, a bromine atom, or phenyl group.

12. The medicament according to claim 1, wherein R¹ is methyl group or propyl group.

13. The medicament according to claim 1, wherein the active ingredient is a substance selected from the group consisting of the following compounds:
5-methyl-2-(1-piperazinyl)benzenesulfonic acid;
5-trifluoromethyl-2-(1-piperazinyl)benzenesulfonic acid;
5-n-propyl-2-(1-piperazinyl)benzenesulfonic acid;
5-phenyl-2-(1-piperazinyl)benzenesulfonic acid;
5-chloro-2-(1-piperazinyl)benzenesulfonic acid;
5-bromo-2-(1-piperazinyl)benzenesulfonic acid;
5-isopropyl-2-(1-piperazinyl)benzenesulfonic acid;
5-cyclohexyl-2-(1-piperazinyl)benzenesulfonic acid;
5-n-propyl-2-(1-homopiperazinyl)benzenesulfonic acid;
5-n-propyl-2-[4-(2,3,4-trimethoxybenzyl)-1-piperazinyl]benzenesulfonic acid; and
5-n-propyl-2-[4-(3,4-dimethoxybenzyl)-1-piperazinyl]benzenesulfonic acid and a salt thereof, and a hydrate thereof and a solvate thereof.

14. The medicament according to claim 13, wherein the active ingredient is a substance selected from the group consisting of the following compounds:
5-methyl-2-(1-piperazinyl)benzenesulfonic acid and
5-n-propyl-2-(1-piperazinyl)benzenesulfonic acid and a salt thereof, and a hydrate thereof and a solvate thereof.

15. The medicament according to claim 1, wherein the active ingredient is 5-methyl-2-(1-piperazinyl)benzenesulfonic acid monohydrate.
